(19) United States
(12) Patent Application Publication
(10) Pub. No.: US 2016/0361563 A1
(43) Pub. Date: Dec. 15, 2016

(54) PORTABLE ELECTRONIC DEVICE

(71) Applicant: UPI SEMICONDUCTOR CORP., Hsinchu County (TW)

(72) Inventor: Kun-Chang WANG, Hsinchu County (TW)

(21) Appl. No.: 14/930,626

(22) Filed: Nov. 2, 2015

(30) Foreign Application Priority Data
Jun. 10, 2015 (TW) 104118796

(51) Int. Cl. A61N 5/06; H05K 5/00

(52) U.S. Cl. CPC A61N 5/0616; H05K 5/0086

(57) ABSTRACT

A portable electronic device includes a main body and a functioning module. The main body has an accommodation space. The main body includes at least one magnetic connecting portion disposed in the accommodation space. The functioning module is detachably accommodated in the accommodation space. The functioning module has a functioning surface and a connecting surface. The connecting surface includes at least one first magnetic piece. When the functioning module is accommodated in the accommodation space, the first magnetic piece is magnetically connected with the magnetic connecting portion.

### RELATED APPLICATIONS

This application claims priority to Taiwanese Application Serial Number 104118796, filed Jun. 10, 2015, which is herein incorporated by reference.

### BACKGROUND

Technical Field

The present disclosure relates to a portable electronic device. More particularly, the present disclosure relates to a portable electronic device with an exchangeable functioning module.

Description of Related Art

With the continuous improvement of living standard, people become more concerned about their appearance care. Thus, portable electronic devices with various cosmetic effects appear on the market consecutively.

Among various choices of portable electronic devices, rejuvenation light is a popular, convenient and lightweight choice. Through the cosmetic effect of the rejuvenation light applied on a user's skin, the user's skin quality can be effectively improved.

However, different rejuvenation lights are required for different cosmetic effects. Thus, users have to buy different rejuvenation lights in order to meet their different cosmetic needs. In addition to increasing the users' financial burden, the users also have to prepare a storage space to accommodate a large number of rejuvenation lights, thus inevitably causing various inconveniences to the users.

### SUMMARY

A technical aspect of the present disclosure provides a portable electronic device for allowing a user to exchange the functioning modules at any time in a simple and easy manner according to actual needs.

According to an embodiment of the present disclosure, a portable electronic device includes a main body and a functioning module. The main body has an accommodation space. The main body includes at least one magnetic connecting portion disposed in the accommodation space. The functioning module is detachably accommodated in the accommodation space. The functioning module has a functioning surface and a connecting surface. The connecting surface includes at least one first magnetic piece. When the functioning module is accommodated in the accommodation space, the first magnetic piece is magnetically connected with the magnetic connecting portion.

According to an embodiment of the present disclosure, the number of the at least one magnetic connecting portion is two. The magnetic connecting portions are symmetrically disposed in the accommodation space relative to an axis. The number of the at least one first magnetic piece is two. The first magnetic pieces are oppositely disposed on the connecting surface.

According to an embodiment of the present disclosure, the main body further includes at least one second magnetic piece. The second magnetic piece is adjacent to the magnetic connecting portion. The second magnetic piece is disposed opposite to the first magnetic piece. A magnetic pole of the second magnetic piece facing the first magnetic piece is the same as a magnetic pole of the first magnetic piece facing the second magnetic piece.

According to an embodiment of the present disclosure, the magnetic connecting portion is a C-shaped track.

According to an embodiment of the present disclosure, the main body has electrode tracks located in the accommodation space, and each of the electrode tracks is disposed around an axis. The functioning module further includes pogo pins. The pogo pins are configured to electrically connect with the electrode tracks respectively.

According to an embodiment of the present disclosure, each of the electrode tracks is in an annular shape.

According to an embodiment of the present disclosure, the main body is ridged between the electrode tracks.

When compared with the prior art, the above-mentioned embodiments of the present disclosure have at least the following advantages:

(1) When the functioning module is installed on the main body, a user only needs to magnetically connect the first magnetic piece of the connecting surface to the magnetic connecting portion of the main body for completing the installation. After the functioning module rotates about the axis relative to the main body until the first magnetic piece slides out of the magnetic connecting portion, the first magnetic piece and the magnetic connecting portion are no longer magnetically connected. At this point, the functioning module can be detached from the main body. In this way, it is simple and easy to install the functioning module to the main body or to detach the functioning module from the main body. Moreover, the user does not need to use any extra tool in the process of installation and detachment, thus providing further convenience to the user.

(2) Through the simple and easy process of installation and detachment, the user can exchange the suitable functioning module at any time according to actual needs. Furthermore, the user only needs to own one single main body, and different functioning modules can be matched for use, thus not only saving money but also providing user convenience.

(3) When installing the functioning module on the main body, the user only needs to magnetically connect the first magnetic piece to any position of the magnetic connecting portion. Therefore, the installation of the functioning module and the main body in the direction of the axis has a lot of flexibility, thus providing further convenience to the user.

### BRIEF DESCRIPTION OF THE DRAWINGS

The disclosure can be more fully understood by reading the following detailed description of the embodiments, with reference made to the accompanying drawings as follows:

FIG. 1 is a partially exploded view of a portable electronic device according to an embodiment of the present disclosure;

FIG. 2 is a bottom view of a functioning module shown in FIG. 1;

FIG. 3 is a partially side view of the portable electronic device shown in FIG. 1;

FIG. 4 is a schematic view of an electrical connection between pogo pins and electrode tracks shown in FIG. 1;

FIG. 5 is a partially enlarged view of a main body shown in FIG. 1;

FIG. 6 is a partially exploded view of a portable electronic device according to another embodiment of the present disclosure; and

FIG. 7 is a bottom view of a functioning module shown in FIG. 6.

### DETAILED DESCRIPTION

Drawings will be used below to disclose embodiments of the present disclosure. For the sake of clear illustration, many practical details will be explained together in the description below. However, it is appreciated that the practical details should not be used to limit the claimed scope. In other words, in some embodiments of the present disclosure, the practical details are not essential. Moreover, for the sake of drawing simplification, some customary structures and elements in the drawings will be schematically shown in a simplified way. Wherever possible, the same reference numbers are used in the drawings and the description to refer to the same or like parts.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

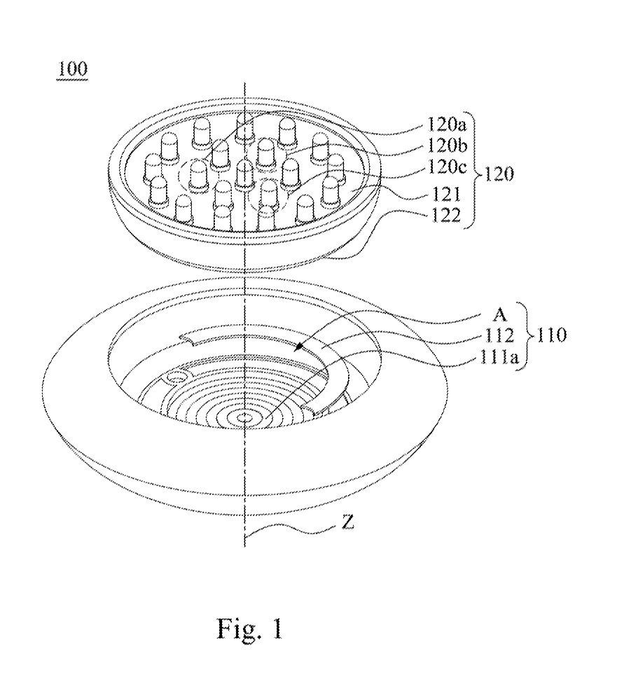

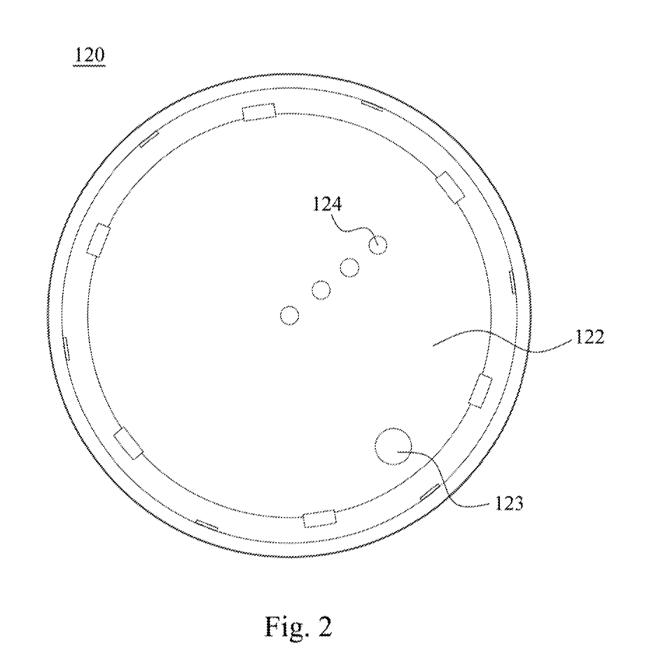

Please refer to FIG. 1 and FIG. 2. FIG. 1 is a partially exploded view of a portable electronic device 100 according to an embodiment of the present disclosure. FIG. 2 is a bottom view of a functioning module 120 shown in FIG. 1. As shown in FIG. 1 and FIG. 2, the portable electronic device 100 includes a main body 110 and a functioning module 120. The main body 110 has an accommodation space A. The main body 110 includes at least one magnetic connecting portion 112. The magnetic connecting portion 112 is disposed in the accommodation space A. The functioning module 120 is detachably accommodated in the accommodation space A. The functioning module 120 has a functioning surface 121 and a connecting surface 122. The connecting surface 122 includes at least one first magnetic piece 123. When the functioning module 120 is accommodated in the accommodation space A, the functioning surface 121 is at least partially exposed from the accommodation space A, and the first magnetic piece 123 is magnetically connected with the magnetic connecting portion 112. When the functioning module 120 rotates about the axis Z relative to the main body 110, the first magnetic piece 123 can be selectively attached to or detached from the magnetic connecting portion 112. After the first magnetic piece 123 is detached from the magnetic connecting portion 112, the functioning module 120 can be detached from the main body 110.

In other words, when the functioning module 120 is installed on the main body 110, a user only needs to magnetically connect the first magnetic piece 123 of the connecting surface 122 to the magnetic connecting portion 112 of the main body 110 for completing the installation. After the functioning module 120 rotates about the axis Z relative to the main body 110 until the first magnetic piece 123 slides out of the magnetic connecting portion 112, the first magnetic piece 123 and the magnetic connecting portion 112 are no longer magnetically connected. At this point, the functioning module 120 can be detached from the main body 110. In this way, it is simple and easy to install the functioning module 120 to the main body 110 or to detach the functioning module 120 from the main body 110. Moreover, the user does not need to use any extra tool in the process of installation and detachment, thus providing further convenience to the user.

Through the simple and easy process of installation and detachment as mentioned above, the user can exchange the suitable functioning module 120 at any time according to actual needs. Furthermore, the user only needs to own one single main body 110, and different functioning modules 120 can be matched for use, thus not only saving money but also providing user convenience.

In practical applications, as shown in FIG. 1, the functioning module 120 further includes units with specific cosmetic effects such as a light emitting unit 120a, a temperature adjusting unit 120b and an vibrating unit 120c. In this embodiment, the light emitting unit 120a, the temperature adjusting unit 120b and the vibrating unit 120c are located on the functioning surface 121. The light emitting unit 120a can emit suitable light beams to the user's skin according to actual needs. The temperature adjusting unit 120b can provide a suitable temperature to the user's skin according to actual needs. The vibrating unit 120c can provide a local massaging effect to the user according to actual needs. It is noted that the aforementioned units included in the functioning module 120 are merely illustrative, but do not intend to limit the claimed scope. A person having ordinary skill in the art may choose which types of units should be included in the functioning module 120 according to actual needs.

Moreover, when installing the functioning module 120 on the main body 110, the user only needs to magnetically connect the first magnetic piece 123 to any position of the magnetic connecting portion 112. Therefore, the installation of the functioning module 120 and the main body 110 in the direction of the axis Z has a lot of flexibility, thus providing further convenience to the user.

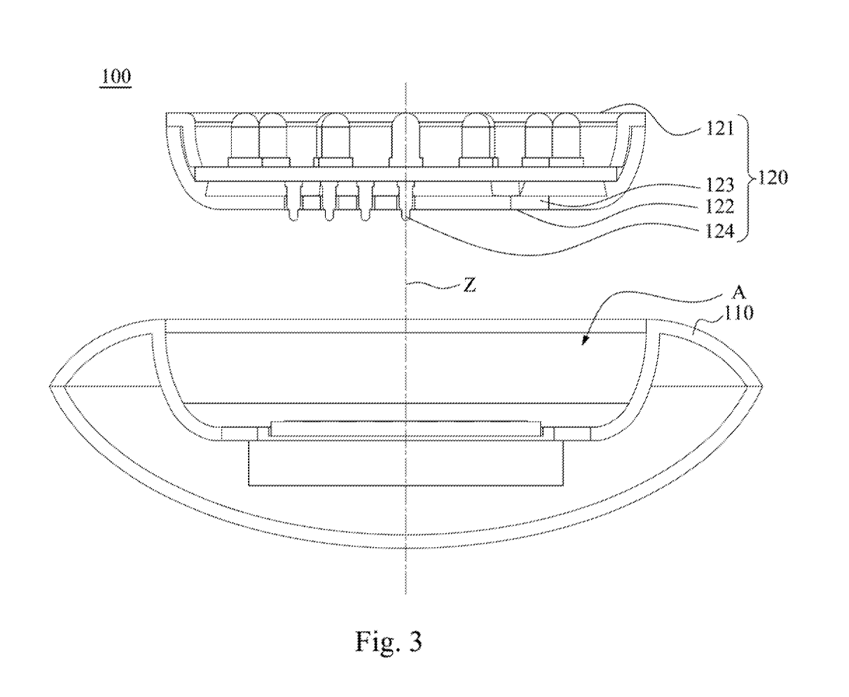

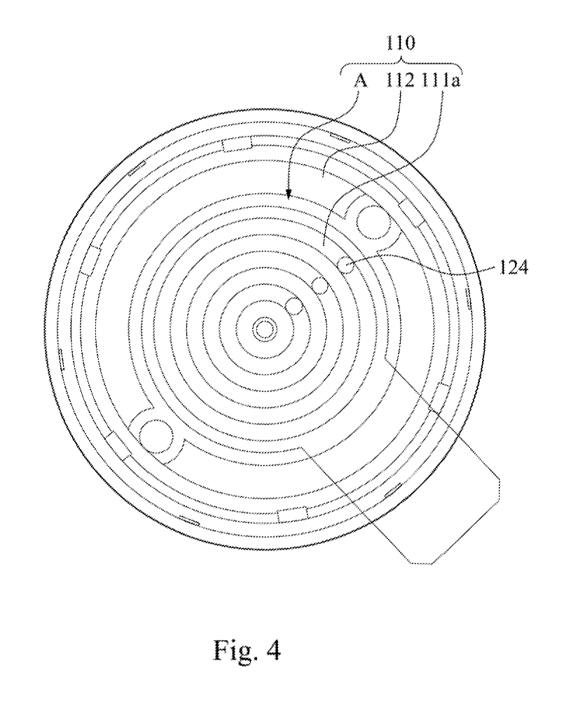

As shown in FIG. 1, the main body 110 has electrode tracks 111a located in the accommodation space A, and each of the electrode tracks 111a is disposed around an axis Z. Please refer to FIG. 3. FIG. 3 is a partially side view of the portable electronic device 100 shown in FIG. 1. As shown in FIG. 2 and FIG. 3, the functioning module 120 further includes pogo pins 124. The pogo pins 124 are located on the connecting surface 122 and are configured to electrically connect with the electrode tracks 111a respectively. Please refer to FIG. 4. FIG. 4 is a schematic view of an electrical connection between the pogo pins 124 and the electrode tracks 111a of FIG. 1. As shown in FIG. 4, each of the pogo pins 124 is electrically connected with the electrode tracks 111a. Through the electrical connection between the pogo pins 124 and the electrode tracks 111a, during the operation of the portable electronic device 100, the main body 110 can transmit electricity and control signals to the functioning module 120, and the functioning module 120 can transmit the data collected to the main body 110 during the operation of the portable electronic device 100.

In this embodiment, as shown in FIG. 1, each of the electrode tracks 111a is in an annular shape. To be further illustrated, when the functioning module 120 is installed on the main body 110, as mentioned above, the first magnetic piece 123 can be magnetically connected to any position of the magnetic connecting portion 112. Since the electrode tracks 111a are disposed around the axis Z and are in annular shapes, no matter which position of the magnetic connecting portion 112 is magnetically connected to the first magnetic piece 123, the pogo pins 124 can still be electrically connected to the corresponding electrode tracks 111a. In this way, the installation of the functioning module 120 and the main body 110 in the direction about the axis Z has a lot of flexibility, thus providing further convenience to the user.

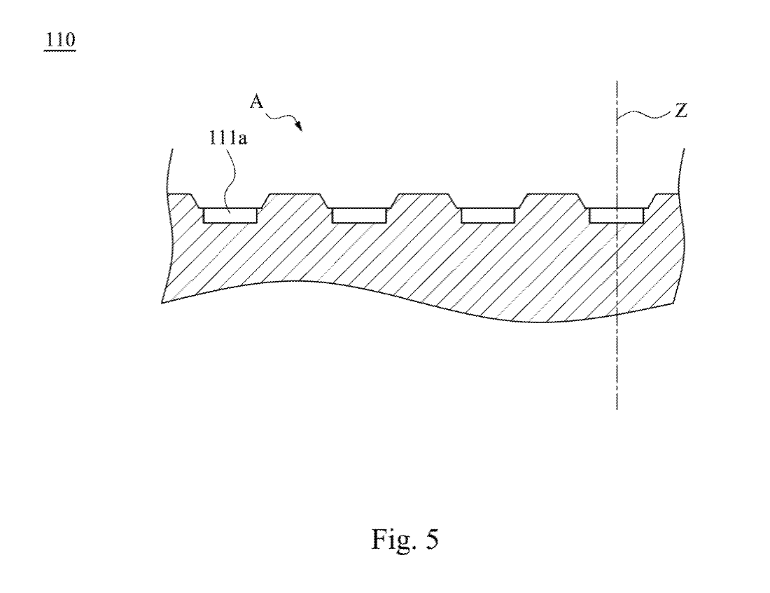

Please refer to FIG. 5. FIG. 5 is a partially enlarged view of the main body 110 shown in FIG. 1. In this embodiment, as shown in FIG. 5, the main body 110 is ridged between the electrode tracks 111a. In this way, even if any conductive foreign object is dropped into the accommodation space A, the conductive foreign object will be supported by the ridged main body 110, thereby preventing the conductive foreign object from electrically connecting with any two of the electrode tracks 111a, thus avoiding the short circuit among the electrode tracks 111a to effectively increase the service life of the portable electronic device 100.

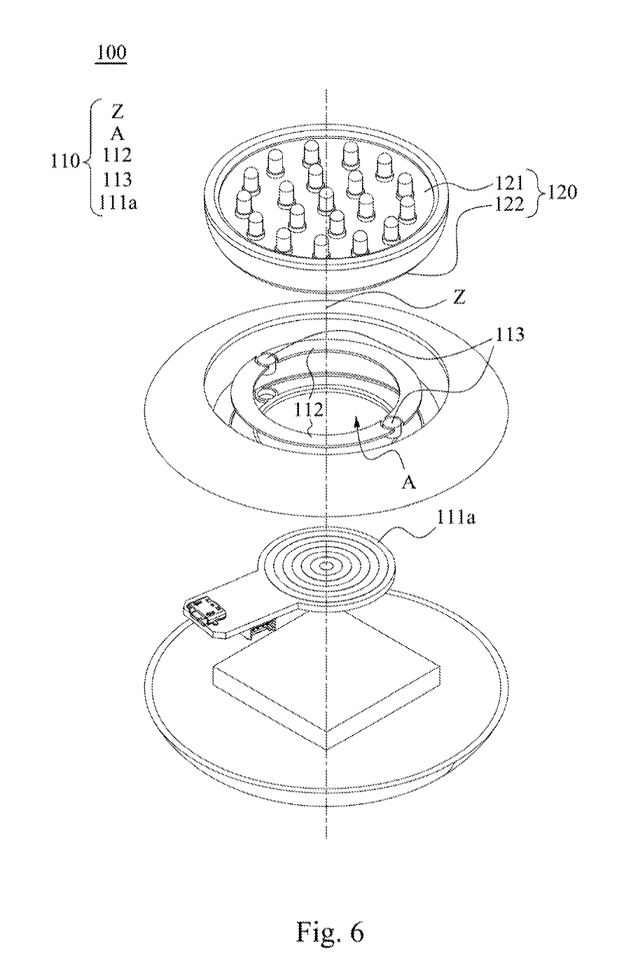

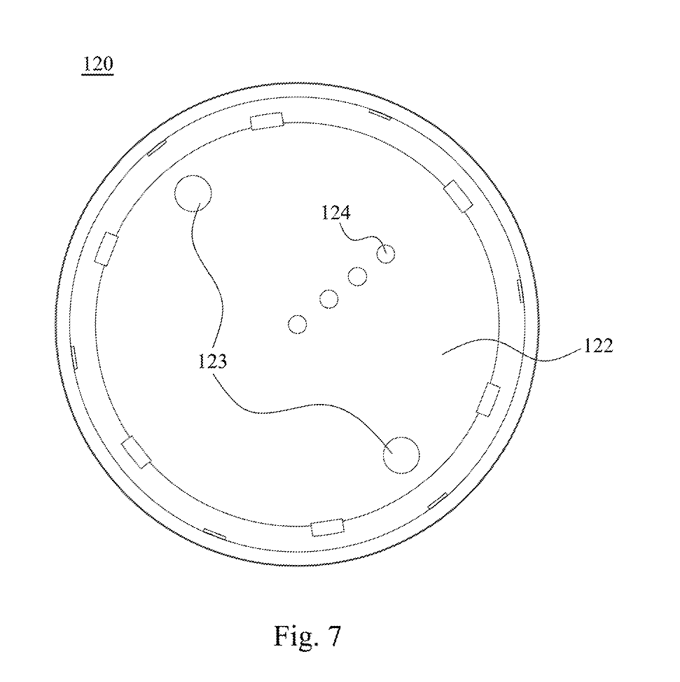

Please refer to FIG. 6 and FIG. 7. FIG. 6 is a partially exploded view of a portable electronic device 100 according to another embodiment of the present disclosure. FIG. 7 is a bottom view of a functioning module 120 shown in FIG. 6. As shown in FIG. 6 and FIG. 7, the main body 110 further includes at least one second magnetic piece 113. In this embodiment, the number of the at least one second magnetic piece 113 is two. One second magnetic piece 113 is located adjacent to the end of the magnetic connecting portion 112. The other second magnetic piece 113 is disposed opposite to the first magnetic piece 123. The magnetic pole of the second magnetic piece 113 facing the first magnetic piece 123 is the same as the magnetic pole of the first magnetic piece 123 facing the second magnetic piece 113. In this way, when the functioning module 120 rotates about the axis Z relative to the main body 110 until the first magnetic piece 123 slides out of the magnetic connecting portion 112, the first magnetic piece 123 and the second magnetic piece 113 will get closer and a magnetic repulsion will be generated due to the same magnetic poles facing to each other. The magnetic repulsion helps to push the functioning module 120 to detach from the main body 110, thus facilitating the user to detach the functioning module 120 from the main body 110 in an easier manner.

In addition, in this embodiment, as shown in FIG. 6, the number of the at least one magnetic connecting portion 112 is two. The magnetic connecting portions 112 are symmetrically disposed in the accommodation space A relative to an axis Z. As shown in FIG. 7, the number of the at least one first magnetic piece 123 is two. The first magnetic pieces 123 are oppositely disposed on the connecting surface 122 of the functioning module 120. To be more specific, the magnetic connecting portions 122 are C-shape tracks respectively, but the present disclosure is not limited thereto.

In sum, when compared with the prior art, the embodiments of the present disclosure mentioned above have at least the following advantages:

(1) When the functioning module is installed on the main body, a user only needs to magnetically connect the first magnetic piece of the connecting surface to the magnetic connecting portion of the main body for completing the installation. After the functioning module rotates about the axis relative to the main body until the first magnetic piece slides out of the magnetic connecting portion, the first magnetic piece and the magnetic connecting portion are no longer magnetically connected. At this point, the functioning module can be detached from the main body. In this way, it is simple and easy to install the functioning module to the main body or to detach the functioning module from the main body. Moreover, the user does not need to use any extra tool in the process of installation and detachment, thus providing further convenience to the user.

(2) Through the simple and easy process of installation and detachment, the user can exchange the suitable functioning module at any time according to actual needs. Furthermore, the user only needs to own one single main body, and different functioning modules can be matched for use, thus not only saving money but also providing user convenience.

(3) When installing the functioning module on the main body, the user only needs to magnetically connect the first magnetic piece to any position of the magnetic connecting portion. Therefore, the installation of the functioning module and the main body in the direction of the axis has a lot of flexibility, thus providing further convenience to the user.

Although the present disclosure has been described in considerable detail with reference to certain embodiments thereof, other embodiments are possible. Therefore, the spirit and scope of the appended claims should not be limited to the description of the embodiments contained herein.

It will be apparent to the person having ordinary skill in the art that various modifications and variations can be made to the structure of the present disclosure without departing from the scope or spirit of the present disclosure. In view of the foregoing, it is intended that the present disclosure cover modifications and variations of the present disclosure provided they fall within the scope of the following claims.

What is claimed is:

1. A portable electronic device, comprising:
a main body having an accommodation space, the main body comprising at least one magnetic connecting portion disposed in the accommodation space; and
a functioning module detachably accommodated in the accommodation space, the functioning module having a functioning surface and a connecting surface, the connecting surface comprising at least one first magnetic piece, wherein, when the functioning module is accommodated in the accommodation space, the first magnetic piece is magnetically connected with the magnetic connecting portion.

2. The portable electronic device of claim 1, wherein the number of the at least one magnetic connecting portion is two, and the magnetic connecting portions are symmetrically disposed in the accommodation space relative to an axis, and the number of the at least one first magnetic piece is two, and the first magnetic pieces are oppositely disposed on the connecting surface.

3. The portable electronic device of claim 1, wherein the main body further comprises:
at least one second magnetic piece adjacent to the magnetic connecting portion, wherein the second magnetic piece is disposed opposite to the first magnetic piece, and a magnetic pole of the second magnetic piece facing the first magnetic piece is the same as a magnetic pole of the first magnetic piece facing the second magnetic piece.

4. The portable electronic device of claim 1, wherein the magnetic connecting portion is a C-shaped track.

5. The portable electronic device of claim 1, wherein the main body has a plurality of electrode tracks located in the accommodation space, and each of the electrode tracks is disposed around an axis, and the functioning module further comprises:
a plurality of pogo pins configured to electrically connect with the electrode tracks respectively.

6. The portable electronic device of claim 5, wherein each of the electrode tracks is in an annular shape.

7. The portable electronic device of claim 5, wherein the main body is ridged between the electrode tracks.
